(19) United States
(12) Patent Application Publication
(10) Pub. No.: US 2011/0152874 A1
(43) Pub. Date: Jun. 23, 2011

(54) BALLOON DILATIONAL CHEST TUBE METHOD AND SYSTEM

(75) Inventor: Drew P. Lyons, Ellettsville, IN (US)

(73) Assignee: Cook Critical Care Incorporated, Bloomington, IN (US)

(21) Appl. No.: 12/643,249

(22) Filed: Dec. 21, 2009

(51) Int. Cl. A61B 17/34

(52) U.S. Cl. 606/108

(57) ABSTRACT

A method and system for inserting a chest tube through the chest wall of a patient into the pleural cavity. An inserter comprises an elongated tubular member and an inflatable balloon positioned at a distal end of the tubular member. A chest tube has a bore extending therethrough, and is sized such that the inserter is receivable in the chest tube bore. The chest tube and inserter are aligned such that the balloon extends distal of the chest tube when the inserter is received in the chest tube bore. An opening is formed through the chest wall to the pleural cavity, and the inserter is advanced into the chest wall opening such that the balloon is positioned across the opening in an uninflated condition. The balloon is inflated to dilate the opening, and the chest tube is advanced into the dilated opening such that the distal end of the chest tube extends through the opening into the pleural cavity.

### BACKGROUND

The present invention relates to the field of chest drainage, and more particularly, to a method and system for drainage of fluid from the pleural space of a patient.

In the human body, the lungs are surrounded by the pleura. The pleura is a serous membrane which folds back upon itself to form a two membrane structure. The two membranes are known as the parietal pleura and the visceral pleura, respectively. The parietal (outer) pleura lines the chest wall, while the visceral (inner) pleura surrounds the lung. The space between the two pleurae layers is known as the pleural space, which space typically contains a thin layer of pleural fluid. This thin layer of fluid provides lubrication to enable the plurae layers to smoothly slide over one another during respiration.

Pleural effusion refers to a condition that occurs when an excess of fluid accumulates in the pleural space. Typically, such accumulation results from chest trauma experienced by the patient. The collection of air in the pleural space results in a condition commonly referred to as pneumothorax. The collection of blood in the pleural space results in a condition commonly referred to as hemothorax. Other fluids that may collect in the pleural space include serous fluid (hydrothorax), chyle (chylothorax), and pus (pyothorax). The presence of excessive amounts of fluids in the pleural space impairs the breathing ability of the patient by limiting the ability of the lungs to expand during inhalation.

In order to drain excess fluid, a chest tube may be inserted into the pleural space. Often the chest tube is inserted utilizing the well-known Seldinger technique. In the Seldinger technique, a needle is initially advanced into the pleural space. A wire guide is inserted through a bore of the needle, and the needle is thereafter removed, leaving the distal end of the wire guide positioned in the pleural space. A series of tapered dilators (such as three) are sequentially advanced (small to large) over the wire guide to dilate the tissue of the chest wall, and form an opening, or stoma, of desired size. After removal of the largest dilator, the chest tube, with inserter/obturator, is placed over the wire guide, and the distal end of the tube is directed into the pleural space.

Although this procedure is generally suitable to enable the physician to properly insert and position the chest tube, the procedure is time consuming and requires the use of multiple devices for its completion, including the multiplicity of tapered dilators. In addition, the necessity to repeatedly insert pointed dilators in the direction of the posterior pleural cavity presents a risk of puncture if each dilation is not carried out properly.

It is desired to provide a method and system for inserting a chest tube in the pleural space that avoids the problems encountered in the art.

### BRIEF SUMMARY

The present invention addresses the problems of the prior art. In one form thereof, the invention comprises a method of inserting a chest tube through the chest wall of a patient into the pleural space. A chest tube and an inserter are provided. The inserter comprises an elongated tubular member and an inflatable balloon positioned at a distal end of the tubular member. The balloon is sized to dilate a portion of the chest wall upon inflation. The chest tube has a bore extending therethrough, and is sized such that the inserter is receivable in the chest tube bore. The chest tube and inserter are aligned such that the balloon extends distal of the chest tube when the inserter is received in the chest tube bore. An opening is formed through the chest wall to the pleural space. The inserter is advanced into the opening such that the balloon is positioned across the opening in an uninflated condition. The balloon is inflated to thereby dilate the opening. The chest tube is thereafter advanced into the dilated opening such that a distal end of the chest tube extends through the opening into the pleural space.

In another form thereof, the invention comprises a method of percutaneously inserting a chest tube through the chest wall of a patient into the pleural space. A needle is advanced through the chest wall such that a tip of the needle extends into the pleural space. The distal end of a wire guide is inserted through the bore of the needle such that the wire guide extends across the chest wall, and the wire guide distal end extends into the pleural space. The needle is removed, leaving the wire guide in place. A chest tube and inserter are provided. The chest tube has a bore extending therethrough, and a plurality of side ports at its distal end. The inserter comprises an elongated tubular member having at least one bore extending therethrough. The inserter further comprises a balloon positioned at the tubular member distal end. The inserter is received in the chest tube bore and aligned therein such that the balloon extends distal of the chest tube, the balloon being inflatable to a diameter at least as large as an outer diameter of the chest tube. The proximal end of the wire guide is inserted into the bore of the inserter, and the chest tube and inserter are advanced over the wire guide such that the balloon lies across the chest wall. The balloon is inflated to dilate a portion of the chest wall. The balloon is thereafter deflated, and the chest tube is advanced along the wire guide such that a distal end of the chest tube extends into the pleural space. The inserter is then withdrawn through the chest tube bore.

In yet another form thereof, the invention comprises a system for use in inserting a chest tube through a chest wall of a patient. The system includes a chest tube having a proximal end, a distal end, a bore extending therethrough, and a plurality of side ports at the distal end. An inserter comprises an elongated tubular member having a proximal end, a distal end, and at least one bore extending therethrough. The inserter further comprises a balloon positioned at the tubular member distal end. The inserter is received in the chest tube bore, and aligned therein such that the balloon extends distal of the chest tube. The balloon is inflatable to a diameter at least as large as an outer diameter of the chest tube, and is structured to dilate a portion of the chest wall upon inflation. The system may also include a wire guide sized to be received in the bore of the inserter, and a needle having a bore sized to receive the wire guide therethrough.

### BRIEF DESCRIPTION OF THE DRAWINGS

FIG. 1 is a side view of a balloon dilational chest tube system according to an embodiment of the present invention, with the balloon in an uninflated condition and illustrating a wire guide passing through the system;

FIG. 2 is a side view of the balloon dilational chest tube system as shown in FIG. 1, with the balloon in an inflated condition;

FIG. 3 is an enlarged cross-sectional view of the balloon dilational chest tube system of FIG. 2, taken along line 3-3 of FIG. 2;

FIG. 4 is a side view of the inserter portion of the balloon dilational chest tube system of FIG. 1;

FIG. 5 is a side view of the chest tube portion of the balloon dilational chest tube system of FIG. 1;

FIG. 6 illustrates a first stage of the chest tube insertion method, showing insertion of a needle tip into the pleural space;

FIG. 7 illustrates the insertion of a tip of a wire guide into the pleural space through the bore of the needle of FIG. 6;

FIG. 8 illustrates the insertion of the chest tube dilational system over the wire guide to a depth such that the uninflated balloon spans the chest wall;

FIG. 9 illustrates the balloon upon inflation, showing the dilation of the chest wall;

FIG. 10 illustrates the insertion of the tip of the chest tube into the pleural space over the inserter and wire guide following deflation of the balloon; and

FIG. 11 illustrates the position of the chest tube following removal of the wire guide and the inserter.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated apparatus, and such further applications of the principles of the invention as illustrated therein being contemplated as would normally occur to one skilled in the art to which the invention relates.

In the following discussion, the terms “proximal” and “distal” will be used to describe the opposing axial ends of the balloon dilational chest tube system, as well the opposing axial ends of component features. The term “proximal” is used in its conventional sense to refer to the end of the balloon dilational chest tube system, or component feature, that is closest to the operator during use. The term “distal” is used in its conventional sense to refer to the end of the balloon dilational chest tube system, or component feature, that is initially inserted into the patient, or that is closest to the patient during use.

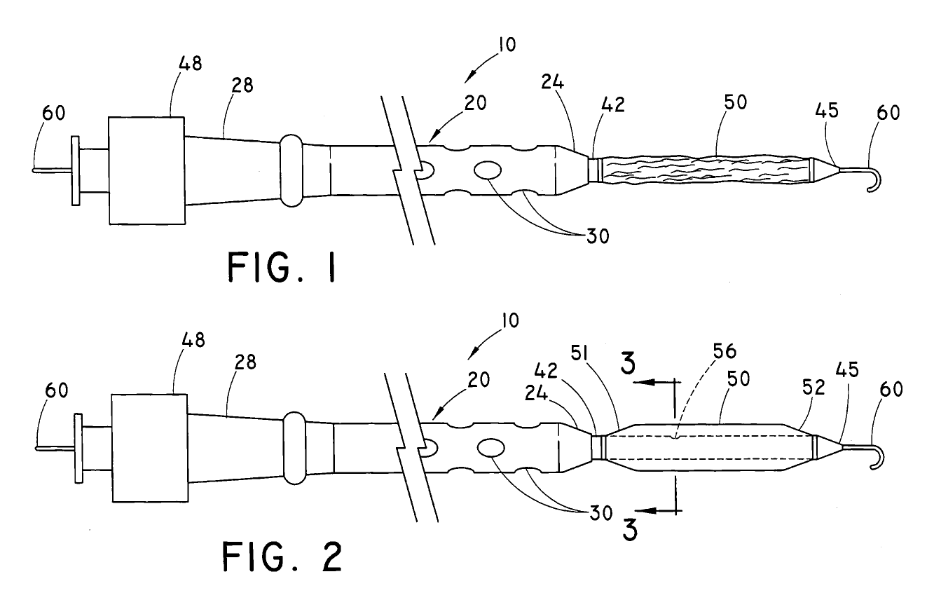

FIG. 1 illustrates a percutaneous balloon dilational chest tube system 10 according to one embodiment of the invention. An inflatable dilational balloon 50, to be further described hereinafter, is shown in an uninflated condition. FIG. 2 illustrates a view of chest tube system 10 as shown in FIG. 1, with the balloon in an inflated condition.

In FIGS. 1 and 2, chest tube system 10 is shown positioned over a conventional J-tipped wire guide 60. Although it is not always necessary to utilize a wire guide for insertion, wire guides are commonly utilized for such purposes, and it is believed that such use facilitates entry of the chest tube. Wire guide 60 can be any conventional type of wire guide commonly used for entry of a chest tube. One wire guide preferred for such use is an Amplatz 0.038 inch (0.97 mm) diameter extra stiff stainless steel wire guide. Those skilled in the art will appreciate that wire guides of other dimensions and stiffness may be substituted in an appropriate case. Balloon dilational chest tube system 10 includes a chest tube 20 (FIG. 5), and a chest tube inserter 40 (FIG. 4) that is received within a longitudinal passageway through chest tube 20 in a manner to be described.

In the embodiment shown, chest tube 20 comprises a hollow elongated tube 22. Elongated tube 22 has a distal end 24 that tapers to open distal tip 25, and may have a flared proximal end 28. Elongated tube 22 has a bore (not shown) extending therethrough suitable for receiving the chest tube inserter, in a manner to be described.

Chest tube 20 may be formed of any materials commonly utilized for such purposes. Typically, chest tube 20 will be formed from a relatively rigid, clear polymer, such as polyvinylchloride. Those skilled in the art will appreciate that other polymers commonly employed for such purposes, such as polyurethane, may be substituted. Chest tube 20 may have any dimensions typically provided with conventional chest tubes. For example, chest tube 20 may have an outer diameter from about 8 to 36 French (2.7 to 12 mm), and an inner diameter from about 0.078 to 0.33 inch (2.0 to 8.4 mm). Chest tube 20 may have a length from about 18 to 41 cm. Typically, smaller French size chest tubes will have a smaller length, and larger French size tubes will have a greater length.

As with conventional chest tubes, chest tube 20 may include one or more radiopaque stripes (not shown) along a length of the chest tube, and if desired, may be provided with a hydrophilic coating along at least the distal portion of its outer surface. A plurality of side ports 30 are provided along the distal end of the elongated tube. Side ports are conventionally provided at the distal end of a chest tube, and side ports 30 may have any conventional size, shape and dimensions. The side ports can be arranged along the distal end of the chest tube in any convenient manner. Typically, such side ports are either arranged linearly or in a spiral pattern. Chest tubes are well known in the art, and to the extent not specifically referenced herein, chest tube 20 may be provided with additional features known to be provided with such tubes.

Chest tube inserter 40 is separately shown in FIG. 4. Inserter 40 is sized to be coaxially carried by and disposed within the bore of elongated tube 22. In the embodiment shown, inserter 40 comprises a hollow elongated tube 42 having a distal end 44 that tapers to distal tip 45. Elongated tube 42 is preferably provided with a conventional connector 48 at its proximal end for engagement with the proximal end 28 of chest tube 20.

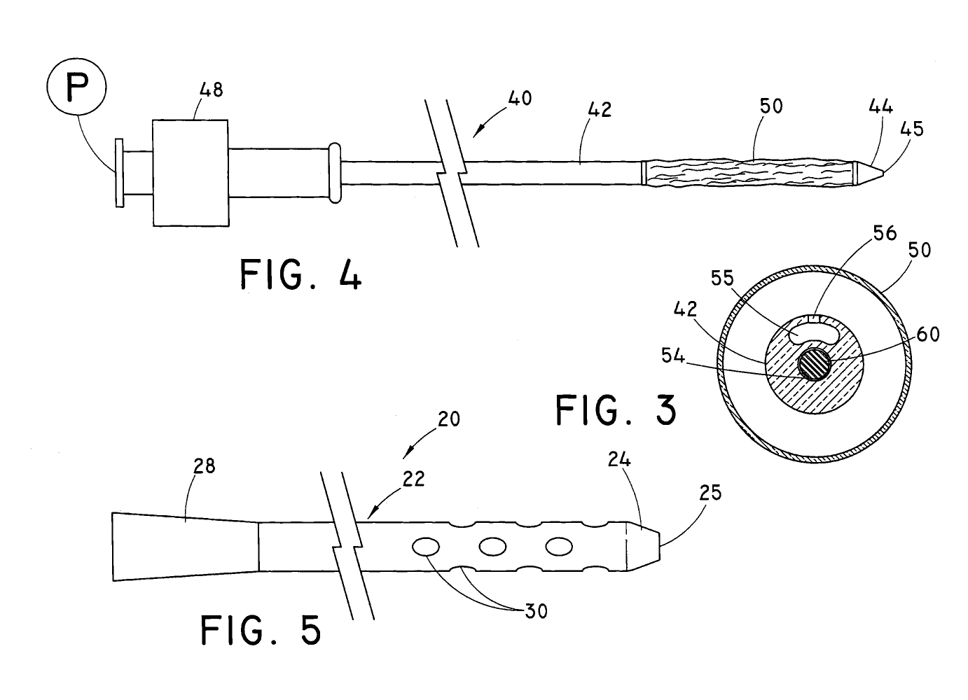

As shown in FIG. 3, elongated tube 42 has a pair of bores 54, 55 extending therethrough. Bore 54 is dimensioned for receiving wire guide 60 therein. Bore 55 is fluidly connected to a supply of pressurized fluid P. Fluid supply P is shown in only a general manner in FIG. 4, as structures for transport of fluid for inflating a catheter balloon are well known in the medical arts. A port 56 in the distal portion of tubular member 42 is open to the interior of balloon 50 to complete the fluid communication of the balloon 50 with the fluid supply P. Preferably, the fluid provided by the fluid supply P is saline solution, although other fluids commonly employed for such purposes may be substituted.

Elongated tube 42 may be formed of a polymeric composition such as vinyl, nylon (polyamide), polyethylene, or other suitable rigid or semi-rigid material. Elongated tube 42 typically has a length from about 20 mm to 45 cm, and an outer diameter from about 9 to 24 French. Bore 54 should, of course, be dimensioned appropriately to allow the wire guide to easily pass therethrough. Thus, for example, with a 0.038 inch (0.97 mm) wire guide as described above, bore 54 should have a diameter of at least about 0.040 inch (1.02 mm). Bore 55 should be dimensioned to allow passage therethrough of sufficient inflation fluid to inflate balloon 50.

Inflatable balloon 50 of inserter 40 is positioned proximal of tapered end 44. Balloon 50 is preferably composed of PET, a polyamide (nylon), or other inelastic high-pressure capable material, and can conveniently have an average burst pressure of, e.g., about seventeen bars. As shown in FIG. 2, balloon 50 is preferably cylindrical in shape when inflated, and has a pair of proximal and distal ends 51, 52, respectively, tapered at an angle of, e.g., about 20-30 degrees with respect to the longitudinal axis of the tubular member 42. The inflated diameter of the balloon 50 is selected in view of the size of opening to be formed in the chest wall. The balloon 50 may, e.g., be about 60 mm long between the ends 51, 52.

The chest tube 20 and inserter 40 are adapted for advancement along wire guide 60 as mentioned above. As indicated above, the purpose of forming an opening through the chest wall is to allow the insertion of a chest tube through the chest wall, so as to establish a passageway for drainage of fluid from the pleural space. It is desirable that the outer diameter of the inserter tubular member 42 be very close to the inside diameter of the chest tube 20. Indeed, these two diameters can possess the same nominal value, that is, the tubular member 42 can have the same nominal 8.5 mm diameter as the nominal 8.5 mm inside diameter of the chest tube 20. The slight resiliency of the tubular member 42 permits this close tolerance; however, if desired, a water-soluble jelly or other lubricant may be applied over the tubular member 42 to ensure that the chest tube 20 does not become stuck on the tubular member 42.

The balloon 50, when inflated, will preferably have a diameter equal to or slightly greater than the outside diameter of the chest tube 20. Therefore, for example, for use with a chest tube 20 having an outside diameter of 12.0 mm, the balloon 50 should have a diameter when inflated of 12.0 mm, or slightly more, perhaps 0.5 to 1.0 mm more.

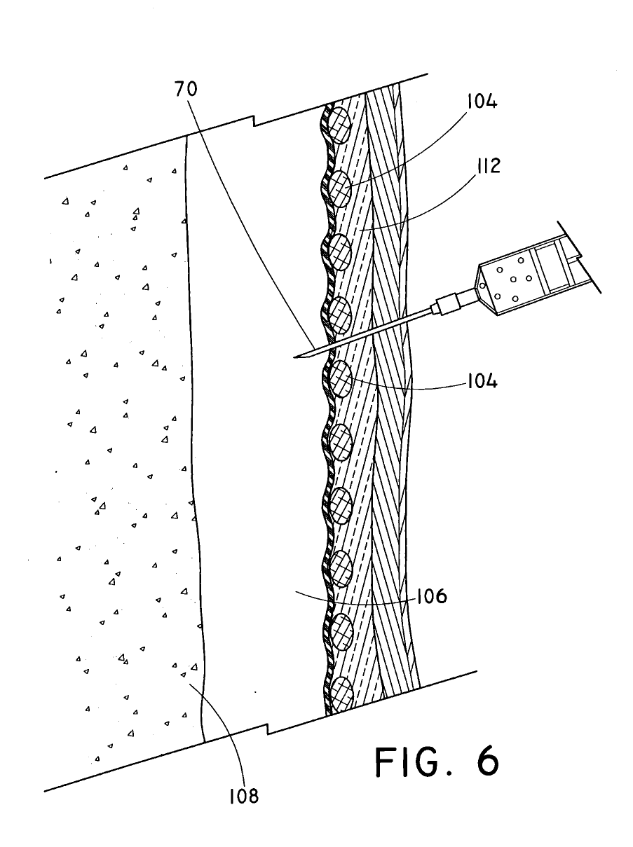

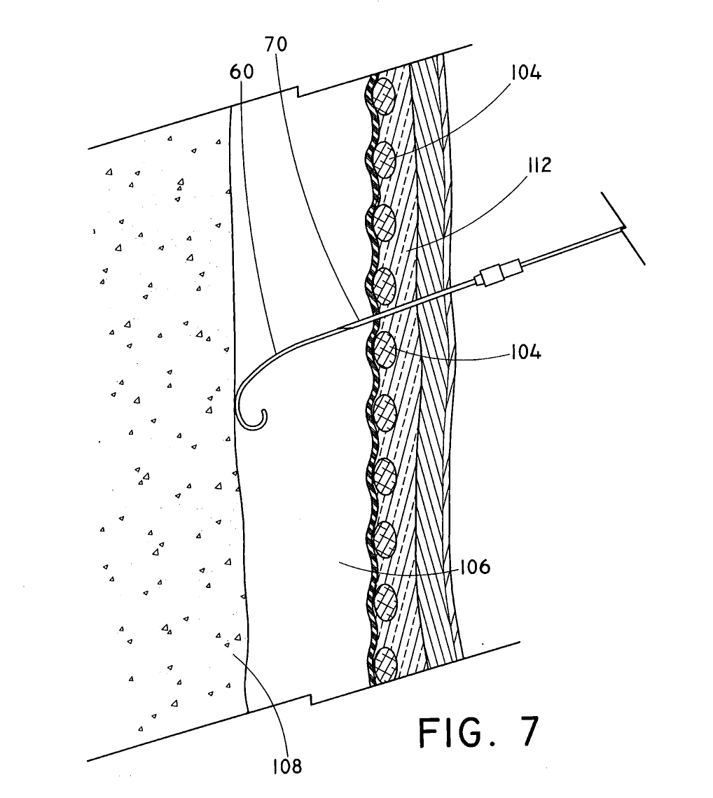

FIGS. 6-11 illustrate various stages of the inventive method for inserting a chest tube through an opening in the chest wall of a patient. Initially, a suitable chest tube insertion site is selected, and a small incision is made utilizing a conventional scalpel (not shown). As shown in FIG. 6, a suitable needle 70, such as an 18 gauge introducer needle, is advanced through the chest wall. In this case, the needle is advanced over the superior border of a rib 104 and through the intercostal muscle 112 into the pleural space 106, being careful not to puncture the lung 108. The end of wire guide 60 is advanced through a bore in needle 70, such that the J-tip of the wire guide enters pleural space 106 (FIG. 7). The needle is then removed, leaving the wire guide in place.

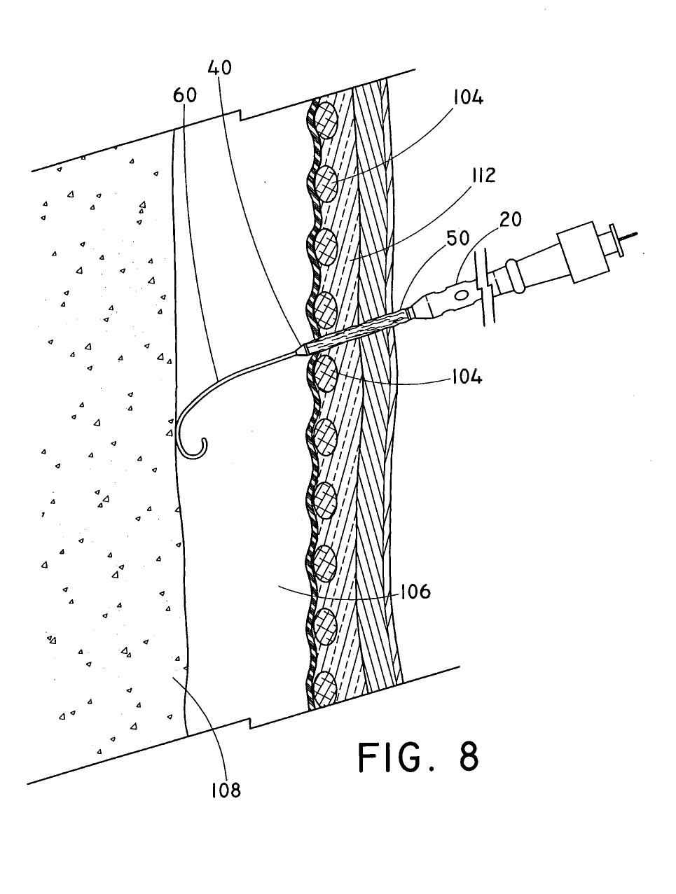

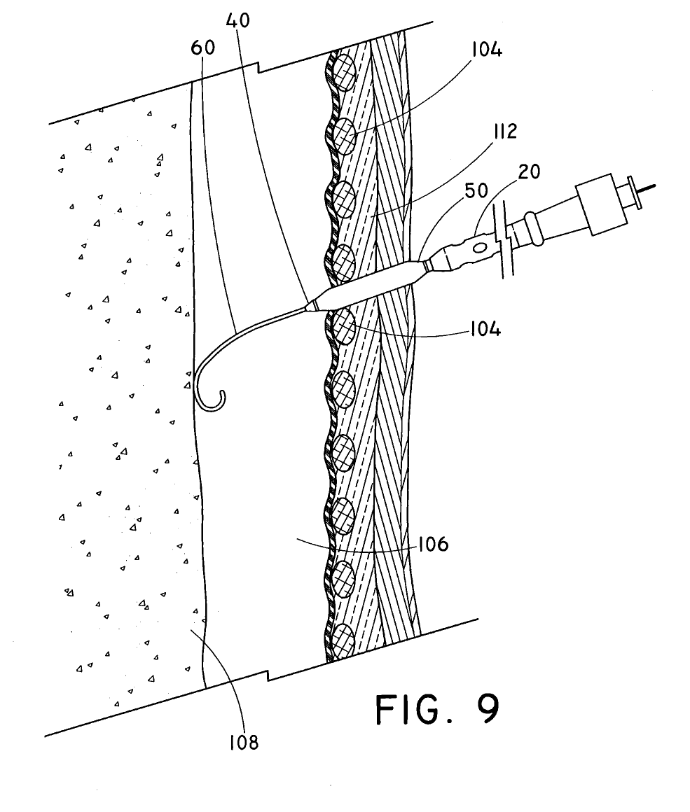

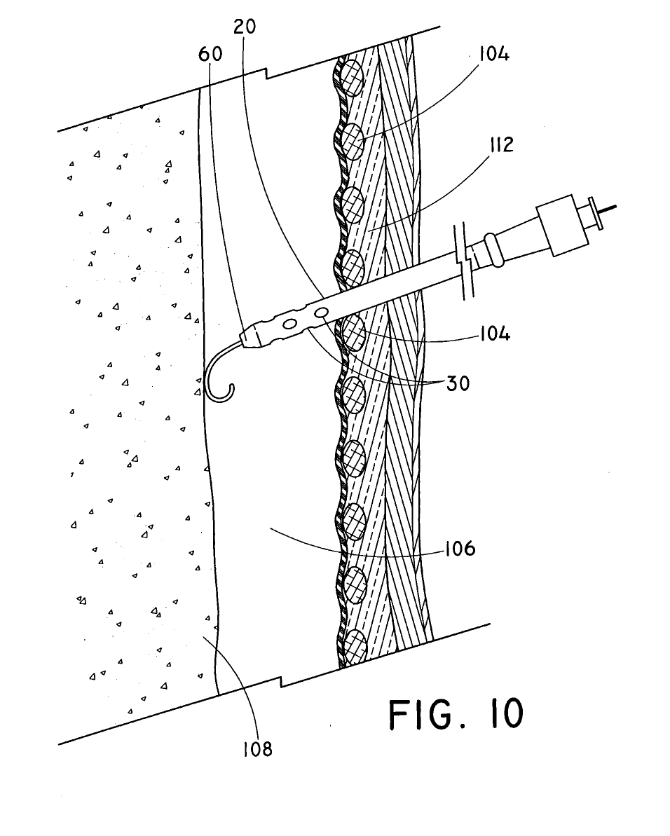

With reference now to FIG. 8, the distal end of the inserter (with the balloon in an uninflated condition) is threaded over the proximal end of the wire guide, and advanced over the wire guide until uninflated balloon 50 lies across the chest wall. Once balloon 50 is properly positioned, the pressurized fluid supply P is activated to provide pressurized fluid through longitudinally extending bore 55 and port 56, thereby inflating balloon 50. Inflation of balloon 50 dilates a portion of the chest wall (FIG. 9), thereby forming an opening in the chest wall of suitable size for receiving the chest tube.

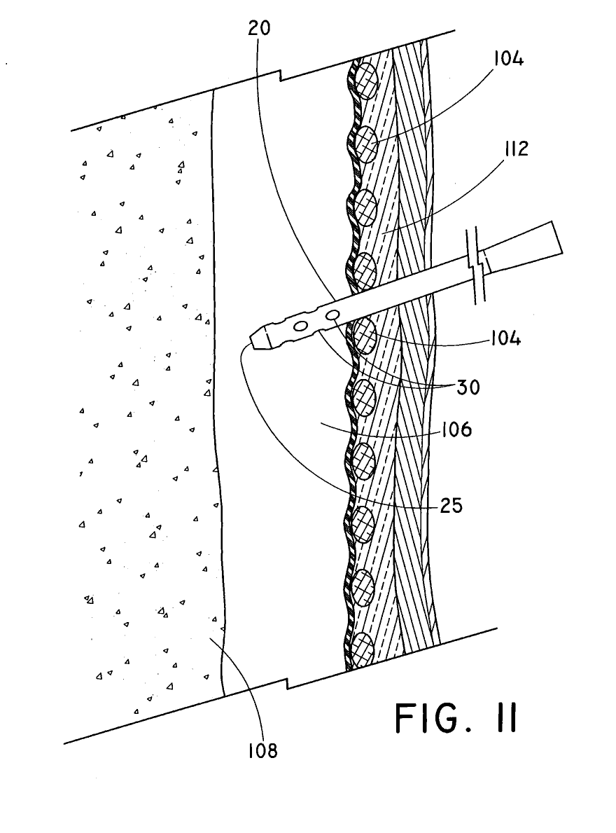

Once the opening is formed in the chest wall, the balloon 50 is deflated by releasing the pressurized fluid. Chest tube 20 is then advanced over the deflated balloon and the wire guide, until the side ports 30 are in the pleural space. This is shown in FIG. 10. The wire guide and chest tube inserter are then withdrawn, leaving the chest tube in place, as shown in FIG. 11. The chest tube may then be secured in typical fashion, e.g. by suturing to the skin. At this time the chest tube is ready for use.

It should be evident from the above discussion that the chest tube system 10 of the present invention can comprise not only the combination of the chest tube 20 and the chest tube inserter 40, but can also include either or both of the wire guide 60 and the hollow needle 70.

The method and system of the present invention possess several advantages over the prior methods and devices for forming or dilating chest wall openings. The present invention allows the opening in the chest wall to be formed and dilated in substantially atraumatic fashion, without enhanced risk of perforating the visceral pleura wall and thereby puncturing the lung. Moreover, the close dilation or slight overdilation of the chest wall opening provided by the balloon 50 facilitates insertion of the chest tube into the opening. The present invention is also relatively less expensive, and simpler in construction and use, than many prior dilation systems.

Any undisclosed details of the construction or composition of the various elements of the disclosed embodiment of the present invention are not believed to be critical to the achievement of the advantages of the present invention, so long as the elements possess the strength or flexibility needed for them to perform as disclosed. The selection of these and other details of construction are believed to be well within the ability of one of ordinary skills in this area, in view of the present disclosure.

It is to be understood, however, that the above described system and method is merely an illustrative embodiment of the principles of this invention, and that other systems and methods may be devised by those skilled in the art, without departing from the spirit and scope of the invention. It is also to be understood that the invention is directed to embodiments both comprising and consisting of the disclosed parts and steps.

1. A method of inserting a chest tube through the chest wall of a patient into the pleural space, comprising the steps of:
providing a chest tube and an inserter; said inserter comprising an elongated tubular member and an inflatable balloon positioned at a distal end of said tubular member, said balloon sized to dilate a portion of said chest wall upon inflation; said chest tube having a bore extending therethrough, and sized such that said inserter is receivable in said bore, said chest tube and said inserter aligned such that said balloon extends distal of said chest tube when said inserter is received in said chest tube bore;
forming an opening through said chest wall to the pleural space;
advancing the inserter into said opening such that said balloon is positioned across said opening in an uninflated condition;
inflating said balloon to thereby dilate said opening; and
advancing the chest tube into the dilated opening such that a distal end of the chest tube extends through said opening into the pleural space.

2. The method of claim 1, further comprising the steps of:
deflating the balloon; advancing the chest tube into the dilated opening over the deflated balloon; and withdrawing the inserter through the chest tube bore.

3. The method of claim 2, further comprising the steps of:
percutaneously inserting a tip of a wire guide through said opening in the chest wall, such that a distal portion of the wire guide extends across the chest wall into the pleural cavity and a proximal portion of the wire guide extends exterior to the patient; and
positioning the inserter over the proximal end of the wire guide for advancement into said opening.

4. The method of claim 3, further comprising the step of passing a needle through said chest wall opening, and inserting the wire guide through a bore in said needle.

5. The method of claim 1, wherein the balloon is inflated to a diameter that is equal to or greater than an outer diameter of the chest tube.

6. The method of claim 3, further comprising the step of securing the chest tube to the patient.

7. The method of claim 3, wherein said inserter comprises a pair of bores, a first one of said bores extending therethrough and sized for receiving said wire guide, a second one of said bores communicating with an interior space of said balloon and configured for transmission of an inflation fluid therethrough.

8. The method of claim 7, wherein said inserter comprises a generally rigid polymer, and said balloon comprises PET.

9. A method of percutaneously inserting a chest tube through the chest wall of a patient into the pleural space, comprising the steps:
advancing a needle through the chest wall such that a needle tip extends into the pleural space;
inserting a distal end of a wire guide through a bore of the needle, such that the wire guide extends across the chest wall, and the wire guide distal end extends into the pleural space;
removing the needle, leaving the wire guide in place;
providing a chest tube and an inserter; said chest tube having a proximal end, a distal end, a bore extending therethrough, and a plurality of side ports at said distal end; said inserter comprising an elongated tubular member having a proximal end, a distal end, and at least one bore extending therethrough, said inserter further comprising a balloon positioned at said tubular member distal end, said inserter received in said chest tube bore and aligned therein such that said balloon extends distal of said chest tube, said balloon inflatable to a diameter at least as large as an outer diameter of said chest tube;
inserting a proximal end of said wire guide into said inserter bore, and advancing the chest tube and inserter over the wire guide such that the balloon lies across the chest wall;
inflating the balloon to dilate a portion of the chest wall;
deflating the balloon;
advancing the chest tube such that a distal end of the chest tube extends into the pleural space; and
withdrawing the inserter through the chest tube bore.

10. The method of claim 9, wherein said balloon is inflated to a diameter that is equal to or greater than a maximum diameter of the chest tube.

11. A system for use in inserting a chest tube through a chest wall of a patient, comprising:
a chest tube having a proximal end, a distal end, and a bore extending therethrough, said chest tube including a plurality of side ports at said distal end; and
an inserter comprising an elongated tubular member having a proximal end, a distal end, and at least one bore extending therethrough, said inserter further comprising a balloon positioned at said tubular member distal end, said inserter received in said chest tube bore and aligned therein such that said balloon extends distal of said chest tube, said balloon inflatable to a diameter at least as large as an outer diameter of said chest tube, said balloon structured to dilate a portion of said chest wall upon inflation.

12. The system of claim 11, further comprising a wire guide sized to be received in said inserter bore.

13. The system of claim 14, further comprising a needle having a bore therein, said needle bore sized to receive said wire guide therethrough.

14. The system of claim 13, wherein said inserter has at least two bores extending therethrough, one of said bores sized for receiving said wire guide, and another bore configured for transmitting an inflation fluid therethrough to an interior of said balloon.
